(19) United States
(12) Patent Application Publication
(10) Pub. No.: US 2014/0165233 A1
(43) Pub. Date: Jun. 12, 2014

(54) METHODS FOR PEST CONTROL

(71) Applicants: Beijing Dabeinong Technology Group Co., Ltd., Beijing (CN); Beijing Green Agrosino Plant Protection Technology Co., Ltd., Beijing (CN); Beijing Dabeinong Technology Group Co., Ltd., Biotech Center, Beijing (CN)

(72) Inventors: Peng CHENG, Beijing (CN); Derong DING, Beijing (CN); Jie PANG, Beijing (CN); Shengbing LI, Beijing (CN); Lijun WANG, Beijing (CN); Jinling SONG, Beijing (CN); Di ZHANG, Beijing (CN); Kaili LI, Beijing (CN); Kangle TIAN, Beijing (CN); Yong TANG, Beijing (CN)

(21) Appl. No.: 14/101,426

(22) Filed: Dec. 10, 2013

(30) Foreign Application Priority Data
Dec. 11, 2012 (CN) 201210533772.2

(51) Int. Cl. C12N 15/82

(52) U.S. Cl. CPC C12N 15/8286
USPC 800/302; 514/4.5

(57) ABSTRACT

Certain embodiments of the present invention provide a method for controlling Athetis lepigone, which comprises contacting Athetis lepigone with Cry1F protein. Aspects of the present invention can achieve control of Athetis lepigone by enabling plants to produce Cry1F protein in vivo, which can be lethal to Athetis lepigone. In still other instances, the method can control Athetis lepigone throughout the growth period of the plants and provide the plants with a full protection. Additionally, the method, in certain embodiments, can be one or more of stable, complete, simple, convenient, economical, pollution-free or residue-free.

### CROSS-REFERENCE TO RELATED APPLICATION

This application claims priority under 35 U.S.C. §119(a)-(d) of Chinese Patent Application No. 201210533772.2 filed Dec. 11, 2012, entitled “Method for Pest Control” which is herein incorporated by reference in its entirety.

### BACKGROUND

Some embodiments of the present invention relate to methods for pest control, such as methods for preventing Athetis lepigone from damaging plants by expressing Cry1F protein therein.

Athetis lepigone belongs to the order of Lepidoptera and the family of Noctuidae. As an omnivorous pest, it sometimes feeds on maize. It can inhabit, in the summer, maize agricultural district of Huang-Huai-Hai region in China, and has also been found in other areas such as in Japan, Korea, Russia and Europe. It can damage aerial roots of maize in topsoil, can eat out maize brace roots and stems, can distort or even kill maize plants. The damaged maize field can show large empty areas or even become sterile if under severe attacks.

Maize is a major food crop in China. On Jul. 9, 2011, the CCTV's “News Broadcast” reported outbreaks of Athetis lepigone in China. From the autumn of 2011 until May 31 2012, several field surveys conducted by the Pest Prevention and Control Laboratory of National Maize Industry found that 2012's Athetis lepigone included a large number of large-size wintering populations with a high density of larvae, indicating that the outbreak of Athetis lepigone is likely to flare up again in Huang-Huai-Hai region. Two methods that can be used to control Athetis lepigone are the agricultural control method and the chemical control method.

The agricultural control method is an integrated and coordinated management of multiple factors for the entire ecosystem of farmland, which regulates crops, pests and environmental factors and establishes a farmland ecosystem conducive to crop growth but unfavorable to Athetis lepigone. For example, the prompt removals of straw, weeds and other coverings from the roots of maize seedlings to a bigger space between maize lines far away from the plants so as to expose the ground, is commonly used, in order to make sure the next step of pesticide spray can directly contact Athetis lepigone. However, since the agricultural control must obey the requirements for crop layout and increasing production, such this method has limited applications and cannot be used as an emergency measure when Athetis lepigone outbreaks.

The chemical control method, also known as the pesticide control method, kills pests by using pesticides. As a means for the comprehensive management of controlling, it can be a fast, convenient, simple and highly cost-effective method. Particularly, it can be used as an emergency practice to reduce the density of Athetis lepigone before damage has occurred. Currently, some measures of the chemical control include poisoned bait, poisoned soil, as well as pesticide drenching and spraying. However, the chemical control has its limitations: its improper use can cause devastating consequences, such as poisoning crops, pest resistance, killing predators and polluting the environment so as to destroy farmland ecosystems; pesticide residues can pose a threat to the safety of local human and livestock; and as Athetis lepigone prefers a moist and dark micro-habitat, it generally hides under coverings such as wheat straws or below the topsoil, making the direct contact between chemicals and Athetis lepigone difficult, which can render the chemical control ineffective.

To overcome one or more of the limitations of the agricultural control method and/or of the chemical control method, researchers have found that, in some instances, inserting genes coding for pesticidal proteins into plant genome can produce pest-resistant plants. Pesticidal protein Cry1F, among a large group of pesticidal proteins, is an insoluble parasporal crystal protein produced by Bacillus thuringiensis.

Cry1F protein, if ingested by pests, can be dissolved in the alkaline environment of the pests' midgut and releases protoxin, a precursor to a toxin. Further, alkaline protease digests the protoxin at its N- and C-terminus and can produce an active fragment, which can subsequently bind to a membrane receptor of epithelial cells of the pests' midgut and can insert itself into the intestinal membrane, resulting in deleterious effects to the pest, such as one or more of cell membrane perforation, disequilibrating the pH homeostasis and/or osmotic pressure across the cell membrane; this can disturb the digestion of the pest, and sometimes eventually lead to the death of the pest.

There are no reports on controlling Athetis lepigone by generating transgenic plants producing a Cry1F protein.

### SUMMARY

Some embodiments of the present invention include providing a pest control method by using transgenic plants expressing Cry1F protein to, for example, control damage caused by Athetis lepigone. In certain embodiments, the method can overcome one or more limitations of the agricultural control method and the chemical control method.

In other embodiments, the method controls (e.g., limits growth or kills) Athetis lepigone, by, for example, contacting (e.g., eating) Athetis lepigone with the Cry1F protein. In certain instances, the Cry1F protein is Cry1Fa protein.

In certain aspects, the transgenic plant expresses Cry1F protein in one or more plant parts, including but not limited to reproductive material, such as seeds, seedlings, and the like.

The Cry1Fa protein can be present in a plant cell expressing the protein, and it can be, in some instances, contacted with Athetis lepigone by ingestion of the plant cell.

Further, in certain embodiments, the Cry1Fa protein is present in a transgenic plant expressing the Cry1Fa protein, and Athetis lepigone contacts with the Cry1Fa protein by ingestion of a tissue of the transgenic plant.

In some embodiments, Athetis lepigone is detrimentally effected, such as, but not limited to the inhibition of growth of Athetis lepigone or death of Athetis lepigone; damage to the plant resulting from Athetis lepigone can, in some instances, be controlled.

In certain embodiments, the transgenic plant can be in any growth period. In other aspects, the tissue of the transgenic plant can be roots, leaves, stems, tassels, ears, anthers or filaments.

The control of the damage of Athetis lepigone to the plant may or may not depend on planting location.

The control of the damage of Athetis lepigone to the plant may or may not depend on planting time.

The plant can be any suitable plant, including but not limited to maize.

In some instances, prior to the step of contacting Athetis lepigone, a transgenic seedling containing a polynucleotide encoding the Cry1Fa protein is planted.

In some embodiments, the amino acid sequence of the Cry1Fa protein comprises an amino acid sequence of SEQ ID NO:1 or SEQ ID NO:2. In still other embodiments, the nucleotide sequence encoding the Cry1Fa protein comprises a nucleotide sequence of SEQ ID NO: 3 or SEQ ID NO:4.

In some instances, the plant can further express at least one of the second nucleotides, which are different from the Cry1Fa protein. In certain aspects, the second nucleotide can encode a Cry-like pesticidal protein, a Vip-like pesticidal protein, a protease inhibitor, lectin, α-amylase or peroxidase. For example, the second nucleotide can encode Cry1Ab protein, Cry1Ac protein, Cry1Ba protein or Vip3A protein. In other examples, the second nucleotide comprises a nucleotide sequence of SEQ ID NO:5 or SEQ ID NO:6. In alternative examples, the second nucleotide is dsRNA, which inhibits an important gene of a target pest.

In some embodiments, the expression of the Cry1F protein in one transgenic plant can be accompanied by the expression of one or more Cry-like pesticidal proteins and/or Vip-like pesticidal proteins. Such co-expression of more than one pesticidal toxins in the same transgenic plant can be achieved by genetic engineering to make the plant containing and expressing genes of interest. In some examples, one plant (the first parent) expresses Cry1F protein by genetic engineering, while another plant (the second parent) expresses a Cry-like pesticidal protein and/or a Vip-like pesticidal protein by genetic engineering; crossing the first parent with the second parent obtains progeny plants expressing all of the genes introduced into the first parent and the second parent.

RNA interference (RNAi) can be a highly specific and efficient degradation of homologous mRNA induced by a double-stranded RNA (dsRNA), which can be highly conserved during evolution. Therefore, some aspects of the present invention can use RNAi to specifically knockout or close the expression of genes of target pests.

### BRIEF DESCRIPTION OF THE DRAWINGS

FIG. 1 is a flow diagram for constructing recombinant cloning vector DBN01-T comprising the nucleotide sequence of Cry1Fa-01 in some embodiments of the present invention;

FIG. 2 is a flow diagram for constructing recombinant expression vector DBN100014 comprising the nucleotide sequence of Cry1Fa-01 in some embodiments of the present invention;

FIG. 3 shows damages to leaves of the transgenic maize plants with inoculation of Athetis lepigone in some embodiments of the present invention;

FIG. 4 shows the development of Athetis lepigone larvae that are inoculated to the transgenic maize plants in some embodiments of the present invention.

### DETAILED DESCRIPTION

Although both Athetis lepigone and Agrotis ypsilon Rottemberg belong to the order Lepidoptera and are of the family Noctuidae, and they have similar targets and close morphology, they are different species in biology. Below include examples of some aspects of Athetis lepigone and Agrotis ypsilon Rottemberg; the descriptions are not necessarily complete and may not be representative of all Athetis lepigone and/or Agrotis ypsilon Rottemberg.

1. Different Feeding Habits.

In addition to severe damage to summer maize, Athetis lepigone also poses a threat to peanut and soybean. Whereas Agrotis ypsilon Rottermberg, as a polyphagous pest, not only harms maize, sorghum and millet, but also causes damage to a broad range of seedlings including larch, pine, Chinese ash and Manchurian walnut in the northeast, Masson pine, fir, mulberry and tea in the south, as well as Chinese red pine, oleaster and other fruit trees in the northwest.

2. Different Geographical Habitations.

Currently Athetis lepigone has been primarily found in Huang-Huai-Hai summer maize district including six provinces of Hebei, Shandong, Henan, Shanxi, Jiangsu and Anhui, a total of 47 cities and 297 towns. Whereas Agrotis ypsilon Rottermberg can be found in places with humid climate and abundant rainfall in China, such as the Changjiang River Valley, southeast coast, and eastern and southern humid areas of the Northeast China.

3. Different Infestation Habits.

Athetis lepigone causes a problem in some of Hebei summer maize districts, particularly in fields interplanted with wheat. Its larvae hide underneath the surrounding crushed wheat straw of maize seedlings or burrow into 2-5 cm of the topsoil to damage maize seedlings. There are normally 1-2 and up to 10-20 larvae per individual seedling. When maize seedlings are at 3-leaf to 5-leaf stage, larvae feed mainly on its stalk base and leave behind round or oval holes of 3-4 mm in size, resulting in the disruption of nutrition transport to leaves and eventually the wilting and death of interior leaves above ground. When targeting maize seedlings of 8-leaf to 10-leaf stage, larvae mainly feed on roots, including aerial roots and main roots, resulting in lodging or even death of the plants. The damaged plants count for 1% to 5% generally and reach up to 15%-20% in more seriously damaged plots. Larvae of Agrotis ypsilon Rottermberg around instars 1-2 can cluster at the top leaves of seedlings day and night and feed on them, but will disperse after instar 3. The larvae are agile and can feign death. They are sensitive to light, and will huddle themselves up when disturbed. During the day they lurk between the layers of wet and dry topsoil, whereas they can excavate from the ground at night to bite seedling plants and drag the injured plants into underground holes, or bite unearthed seeds. Upon the stalk of seedlings becomes harder, they will feed on fresh leaves and growing points. However, when lack of food or finding next overwintering sites, migration occurs to them.

4. Different Morphological Features

1) Different morphology of eggs: Athetis lepigone's eggs are steam-bun-shaped with a longitudinal ridge. Newly laid eggs are yellow-green and turn into khaki at later stages. Agrotis ypsilon Rottermberg's eggs are also steam-bun-shaped but with cross carina. The newly laid eggs are creamy white and gradually becoming yellow, and a black spot would emerge on one top of the eggs before hatch.

2) Different morphology of larvae: Athetis lepigone's mature larvae are about 20 mm long with pale yellow body and brown head. Newly hatched larvae are 14-18 mm in length and yellow-gray or dark-brown in colour. The salient features thereof are a dark brown speckle in inverted triangle shape on individual somites and two brown dorsal lines from dorsal abdomen to the thoracic segment. By contrast, the larvae of Agrotis ypsilon Rottermberg are cylinder-shaped, and the mature larvae thereof are 37-50 mm long and brown headed with irregular dark brown reticulate stripes. They have taupe or fuscous body and rough surface dotted with different-sized particles. Their dorsal line, sub dorsal line and spiracular line are all black brown, the prothorax is fuscous, the pygidium is tawny with two distinct dark brown vertical bands, and the baenopoda and prolegs are tawny.

3) Different morphology of pupae: the pupae of Athetis lepigone are about 10 mm in length, having a fawn body color at early stage then gradually turning brown, and mature larvae pupate underground in the cocoon. By contrast, Agrotis ypsilon Rottermberg's pupae are 18-24 mm in length and bright auburn in color. The mouthpart is lined up with the end of wing buds, both reaching the end of the fourth abdominal segment. The central of the fore part of 4-7 abdominal segments is dark brown with coarse speckles, and has tinny bilateral speckles extended to the spiracle. The anterior part of 5-7 abdominal segments also has tinny speckles. The end of abdomen has a pair of short butt-spines.

4) Different morphology of imago: adult Athetis lepigone is 10-12 mm long and 20 mm with wingspan. The female is slightly larger than the male. Its head, thorax and abdomen are taupe. Its forewings are also taupe but with darker markings, fuscous interior and exterior borderlines, annular markings of a black spot and small reniform patterns. Black dots are present on the edge of the outer concave with a white spot. The exterior borderline is wavy; the edge of wings has a black spot. Its hindwings are white and slightly brown, and gradually becomes fuscous at the edge. Its abdomen is taupe. The valvae of male genitalia is half-wide opening, the dorsal margin is concave with a protruding uncus at the middle, and the aedaeagus has inside spiny needles. By contrast, adult Agrotis ypsilon Rottermberg is 17-23 mm in length, 40-54 mm with wingspan. Its head and the back of thorax are fuscous, and the feet are brown. The forefoot tibia and tarsus edge are taupe, and the terminus of every segment of mid- and meta-legs has taupe annular bands. Its brown forewings have black brown anterior areas, fuscous outer borders, light brown base lines and double-lined black wavy endo-transverse lines. There is one round gray speck within black annular bands. Reniform annular shape is black and has black edge. The middle of outside of the reniform annular shape has wedge-shaped black annular shape, which reaches external transverse lines. The mid-transverse line is fuscous wavy shape. The double-lined wavy external transverse lines are brown. The sub-external borderline is irregular saw-tooth shape and gray, the middle of the inner border of which has three pointed teeth. There are small black dots on each vein between sub-external borderline and external transverse line. The outer borderline is black. The color between external transverse line and sub-external borderline is light brown. The color out of sub-external borderline is black brown. The hindwings are hoar. The longitudinal vein and borderline are brown. The back of abdomen is gray.

5. Different growth habit and breakout pattern. Athetis lepigone's larvae have 6 instars lasting about 18 days and they have a strong stress resistance. The breakout of adults has two distinct peaks. The first one occurs before the beginning of July while the second one is from the mid-July to mid-August. The adults have a strong reproductive capacity: each female has a production of 300-500 eggs in average and the egg-laying period lasts 3-7 days, while the hatching rate can reach up to 100%. They cause more damage in maize field rotating planting after cotton than continuous planting, covered with wheat bran than without wheat bran, late sowing than early sowing, and having high field humidity than having low field humidity. Athetis lepigone favours dark and moist environment and often hides under the straw or soil, causing a great inconvenience for pesticides spraying. In contrast, Agrotis ypsilon Rottermberg has 3-4 generations in one year. Mature larvae or pupae overwinter in the soil and imagoes start to appear in March. Generally, two moth peaks will occur: one in late March and the other in mid-April. Adults are inactive during the day and become active at dusk till midnight. They have phototaxis and favour sour, sweet, wing fermentations, and various kinds of nectars. The larvae go through six instars: at instars 1 and 2, larvae hide inside the weeds or interior leaves of plants, feeding themselves day and night but with little appetite, and thus cause little damages; after instar 3, they hide under the topsoil during the day and come out for food at the night; at instars 5 and 6, larvae start to have an significantly-increased appetite and each individual can break down 4-5 seedlings in average, up to 10 in extreme cases; and since instar 3, their pesticide resistance significantly increases. The severest damage caused by the first generation of larvae occurs between the end of March and the mid-April. Generations occur from October to April of the next year and do damages. The number of generations in a year varies geographically: 2-3 generations in the northeast, 2-3 generations in the north of the Great Wall, 3 generations in the area between the south of the Great Wall and the north of the Yellow River, 4 generations in the area between the south of Yellow River and Yangtze River, 4-5 generations in the south of Yangtze River, and 6-7 generations in the tropical area in south Asia. However, regardless of the difference in the number of generations in one year, the severest damage is always caused by the larvae of the first generation. Imagoes of southern overwintering generation appear in February. However, the eclosion peak normally occurs from the end of March to the middle of April in most of the country except Ningxia and Inner Mongolia, in which it occurs at the end of April. The Imagoes of Agrotis ypsilon Rottermberg are more likely to start eclosion from 15:00 to 22:00. They lurk under debris and crack during the day and become active after dusk, flying and foraging. After 3-4 days, they start mating and laying eggs. The eggs are mainly laid on the short, high-density weeds and seedlings and sometimes in dead leaves and cracks. Most eggs are near the ground. Each female can lay 800-1000 eggs, or even up to 2000 during their oviposition period of about 5 days. The larval stage consists of 6 instars, and some individuals can reach 7-8 instars. The larval stage varies at different places, but normally takes 30-40 days for the first generation. Once fully matured, they develop into pupae in a soiled chamber around 5 cm underground and the pupal stage is 9-19 days. High temperature is harmful for the development and reproduction of Agrotis ypsilon Rottemberg, thus fewer of them appear during the summer. The optimum survival temperature is 15° C.-25° C. The mortality of Agrotis ypsilon Rottemberg's larvae increases when the temperature of winter is too low, and decreases at places where is low terrain, humid and have abundant rainfall. Additionally, conditions conducive to oviposition and larval feeding such as abundant autumn rainfall, high soil moisture and overgrown weed may lead to an outbreak in the next year. However, excessive rainfall and too much moisture are bad for larval development as first-instar larvae can drown very easily in such environment. Regions having 15-20% soil moisture content during the peak period of oviposition would suffer severer damages. Sandy loam soil is more adapted than clay soil and sandy soil to the reproduction of Agrotis ypsilon Rottemberg, due to its better water permeability and quick draining.

Collectively, it is evident that Athetis lepigone and Agrotis ypsilon Rottemberg are two distinct species of pests and cannot crossbreed.

In the present disclosure, the genome of plants, plant tissues or plant cells refers to any genetic material in the plants, tissues or cells, including nucleus, plastid and mitochondrial genome.

In the present disclosure, polynucleotides and/or nucleotides constitute a complete “gene”, and encode a protein or polypeptide in desired host cells. The skilled person in the art would readily recognize that the polynucleotides and/or nucleotides can, in some instances, be placed under the control of regulatory sequences of target hosts.

DNA normally exists in a form known as double-stranded structure. In this arrangement, one strand is complementary with another strand, and vice versa. DNA generates other complementary strands during replication in plants, thus the present invention includes use of the polynucleotides exemplified in the sequence list and their complementary strands. “Coding strand” commonly used in the art refers to the strand binding to the antisense strand. In order to express proteins in vivo, one strand of DNA is typically transcribed into a complementary strand as mRNA, which is used as a template to be translated into protein. In fact, mRNA is transcribed from the “antisense” strand of DNA. “Sense” or “encoding” strand has a series of codons (one codon contains three nucleotides, which encodes a specific amino acid), and the strand can be used as an open reading frame (ORF) and be transcribed into a protein or peptide. The present invention also encompasses RNA and peptide nucleic acid (PNA), which have considerable functions as the exemplified DNA.

In some embodiments, the nucleic acid molecules or fragments thereof hybridize to Cry1Fa gene of the present invention under stringent conditions. Any conventional nucleic acid hybridization or amplification method can be used to identify the presence of the Cry1Fa gene. The nucleic acid molecules or fragments thereof in certain cases can specifically hybridize to other nucleic acid molecules. In certain instances, if two nucleic acid molecules can form an antiparallel double-stranded nucleic acid structure, then these two nucleic acid molecules can specifically hybridize to each other. If two nucleic acid molecules exhibit complete complementarity, one nucleic acid molecule is called the “complement” of the other nucleic acid molecule. When every nucleotide of one nucleic acid molecule is complementary to the corresponding nucleotide of another nucleic acid molecule, the two nucleic acid molecules are called to exhibit “complete complementarity”. If two nucleic acid molecules can hybridize to each other at an efficiently stable status, and bind to each other after annealing under at least conventional “low stringency” conditions, these two nucleic acid molecules are called “minimal complementarity”. Likewise, if two nucleic acid molecules can hybridize to each other at an efficiently stable status, and bind to each other after annealing under conventional “high stringency” conditions, these two nucleic acid molecules are called to have “complementarity”. Deviation from complete complementarity is acceptable as long as such deviation does not completely prevent the two molecules from forming a double-stranded structure. In order to ensure that a nucleic acid molecule can be used as a primer or probe, its sequence must have sufficient complementarity so that it can form a stable double-stranded structure in particular solvents and salt concentrations.

In the present disclosure, a substantially homologous sequence is a nucleic acid molecule, which, under highly stringent conditions, can specifically hybridize with the matched complementary strand of the other nucleic acid molecule. The stringent conditions suitable for DNA hybridization, e.g., processing with 6.0× sodium chloride/sodium citrate (SSC) at about 45° C., and then washing with 2.0×SSC at 50° C., would be well known to the skilled person in the art. For example, during the wash step the salt concentration can be selected from a low stringency condition of about 2.0×SSC to a highly stringent condition of about 0.2×SSC at 50° C. In addition, the temperature in the wash step can be selected from a low stringency condition of room temperature about 22° C. to a highly stringent condition of about 65° C. Both temperature and salt concentration can be changed, or one can remain intact while another one is changed. In some embodiments, the stringent conditions according to the invention are: specific hybridization with SEQ ID NO: 3 or SEQ ID NO: 4 in a 6×SSC, 0.5% SDS solution at 65° C., and then membrane washing with 2×SSC, 0.1% SDS and 1×SSC, 0.1% SDS once each.

Therefore, sequences having pest-resistant activity and capable of hybridizing with SEQ ID NO: 3 and/or SEQ ID NO: 4 under stringent conditions are encompassed by some embodiments of the present invention. These sequences are at least of about 40%-50% homology to the sequences of the present invention, about 60%, 65% or 70% homology, or even at least of about 75%, 80%, 85%, 90%, 91%, 92%, 93%, 94%, 95%, 96%, 97%, 98%, 99% or greater homology to the sequences of the present invention.

The genes and proteins encompassed by some embodiments of the present invention include not only the specifically exemplified sequences described herein, but also the portions and/fragments (including those having internal and/or terminal deletions in comparison with the full-length proteins), variants, mutants, substitutes (proteins with substituted amino acids), chimeric and fusion proteins thereof having the pesticidal activity of the exemplified proteins described herein. The “mutants” or “variants” refer to nucleotide sequences encoding the same protein or equivalent protein with the pesticidal activity. The “equivalent protein” refers to a protein presenting the same or substantially the same biological activity of resistance to Athetis lepigone as the claimed proteins.

The “fragment” or “truncation” of the DNA or protein sequences described in the present invention refers to a part or an artificially modified form (such as sequences suitable for plant expression) of the original DNA or protein sequences (nucleotides or amino acids). The length of the above sequences can be variable, but it should be sufficient to ensure the protein (encoded) as a pest toxin.

Genes can be modified as gene variants by standard techniques. For example, the technology of point mutation is well known in the art. Another example based on U.S. Pat. No. 5,605,793 (which is herein incorporated by reference in its entirety) describes a method that DNA can be reassembled to generate other molecular diversity after random fracture. Commercially manufactured endonucleases can be used to make fragments of full-length genes, and exonucleases can be used according to standard procedures. For example, enzymes such as Bal31 or site-directed mutagenesis can be used to systematically remove nucleotides from the end of these genes. A variety of restriction endonucleases can also be used to obtain genes that encode active fragments. Proteases can also be used to obtain active fragments of these toxins directly.

In certain embodiments of the present invention, equivalent proteins and/or genes encoding these equivalent proteins can be derived from B.t. isolates and/or DNA libraries. There are various ways to obtain the pesticidal proteins of the present invention. For example, antibodies of pesticidal proteins disclosed and claimed by the present invention can be used to identify and isolate other proteins from a mixture of proteins. In particular, antibodies may be produced by the most constant and the most different parts from other B.t. proteins. By immunoprecipitation, enzyme-linked immunosorbent assay (ELISA) or western blot, these antibodies can be used to specifically identify equivalent proteins with characteristic activities. Standard procedures in the art can be used to prepare antibodies of the proteins or equivalents or fragments thereof disclosed in the present invention. Also, the genes encoding these proteins can be obtained from microorganisms.

Due to the redundancy of genetic codes, a variety of different DNA sequences can encode the same amino acid sequence. The skilled person in the art would be able to generate alternative DNA sequences to encode the same or substantially the same protein. These different DNA sequences are included within the scope of certain embodiments of the present invention. The “substantially the same” sequences including fragments with pesticidal activity, refer to sequences with amino acid substitution, deletion, addition or insertion but the pesticidal activity thereof is not essentially affected.

In some embodiments of the present invention, the substitutions, deletions or additions in amino acid sequences can be obtained using any suitable technique, such as conventional techniques in the art. In some instances, the alterations of amino acid sequences are: a slight change of characteristics, i.e., conservative amino acid substitutions that do not significantly affect folding and/or activity of proteins; a short deletion, usually of 1-30 amino acids; a small amino- or carboxyl-terminal extension, such as an amino-terminal extension of a methionine residue; a small peptide linker with a length of about 20-25 residues for example.

Examples of conservative substitutions can be selected from the following groups of amino acids: basic amino acids, such as arginine, lysine and histidine; acidic amino acids, such as glutamic acid and aspartic acid; polar amino acids, such as glutamine, asparagine; hydrophobic amino acids, such as leucine, isoleucine and valine; aromatic amino acids, such as phenylalanine, tryptophan and tyrosinel; and small-molecule amino acids, such as glycine, alanine, serine, threonine and methionine. Sometimes, the amino acid substitutions without changing specific activities are known in the art, and they have been, for example, described in “Protein” by N. Neurath and R. L. Hill in 1979, published by Academic Press, New York. Some substitutions are Ala/Ser, Val/Ile, Asp/Glu, Thu/Ser, Ala/Thr, Ser/Asn, Ala/Val, Ser/Gly, Tyr/Phe, Ala/Pro, Lys/Arg, Asp/Asn, Leu/Ile, Leu/Val, Ala/Glu and Asp/Gly, and opposite substitutions thereof.

These substitutions can, in some instances, occur outside the regions that play important roles on the molecular functions but still produce an active polypeptide. For the polypeptides according to some aspects of the present invention, amino acid residues that are necessary for their activity and thus are selected not to be substituted can be identified through any suitable method known in the art, such as site-directed mutagenesis or alanine scanning mutagenesis (referring to Cunningham and Wells, 1989, Science 244: 1081-1085). The latter technique is to introduce mutation(s) to each positive charged residue in a molecule and detect pest-resistant activity of the resulting mutants, and then to determine which amino acid residues are important for the activity of the molecule. Substrate-enzyme interaction sites can be identified by the analysis of their three-dimensional structures which can be determined by nuclear magnetic resonance analysis, crystallography or photoaffinity labeling, etc (referring to de Vos et al., 1992, Science 255: 306-312; Smith et al., 1992, J. Mol. Biol. 224: 899-904; Wlodaver et al., 1992, FEBS Letters 309: 59-64).

In some embodiments of the present invention, the Cry1F proteins include, but not limited to, Cry1Fa2, Cry1Fa3, Cry1Fb3, Cry1Fb6 or Cry1Fb7 proteins, pesticidal fragments or functional regions that are at least 70% homologous to the amino acid sequences of the above-mentioned proteins, and they have the pesticidal activity to Athetis lepigone.

Therefore, amino acid sequences with certain homology to SEQ ID NO: 1 and/or 2 are also included in some aspects of the present invention. The homology/similarity/identity of these sequences to the sequences of some aspects of the present invention can, in some instances, be greater than 60%, greater than 75%, greater than 80%, greater than 90% or can be greater than 95%. Also, polynucleotides and proteins of certain aspects of the present invention can be defined by a more particular range of identity and/or similarity and/or homology, for example, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity and/or similarity and/or homology to the exemplified sequences of certain aspects of the present invention.

The regulatory sequences described in some embodiments of the present invention include, but are not limited to, promoters, transit peptides, terminators, enhancers, leader sequences, introns and other regulatory sequences that can sometimes be operatively linked to the Cry1F protein.

The promoters can include those expressible in plants; the “promoters expressible in plants” refer to the promoters that ensure expression of the coding sequences connected thereto in plant cells. The promoters expressible in plants can be constitutive promoters. Examples of the promoters directing constitutive expression in the plants include, but are not limited to, 35S promoter from the cauliflower mosaic virus, Ubi promoter, promoter of rice GOS2 gene, etc. Alternatively, the promoters expressible in plants can be tissue-specific, i.e., the expression of coding sequences directed by such promoters in some plant tissues, such as green tissues, is higher than that in other tissues, as determined by routine RNA tests, e.g., PEP carboxylase promoter. Alternatively, the promoters expressible in plants can be wound-inducible promoters. Wound-inducible promoters or promoters directing wound-induced expression patterns refer to that the expression of coding sequences regulated by such promoters is significantly higher in the plants that suffer from mechanical wound or wound caused by pest chewing than in plants under normal growth conditions. Examples of the wound inducible promoters include, but are not limited to, promoters of protease inhibitor genes of potato and tomato (pin I and pin II) and of protease inhibitor gene of maize (MPI).

The transit peptides, also known as secretory signal sequences or guide sequences, direct transgenic products to specific organelles or cellular compartments. The transit peptides can be heterologous for the target proteins. For example, the sequences encoding the chloroplast transit peptide are used to target chloroplast, or ‘KDEL’ retaining sequences are used to target endoplasmic reticulum, or CTPP of barley lectin gene are used to target vacuoles.

The leader sequences include, but are not limited to, leader sequences of small RNA viruses, such as EMCV leader sequence (5′-terminal noncoding region of EMCV (encephalomyocarditis virus)); potyvirus leader sequences, such as MDMV (maize dwarf mosaic virus) leader sequence; human immunoglobulin heavy-chain binding protein (BiP); untranslated leader sequence of mRNA of coat protein of alfalfa mosaic virus (AMV RNA4); and tobacco mosaic virus (TMV) leader sequence.

The enhancers include, but are not limited to, cauliflower mosaic virus (CaMV) enhancer, figwort mosaic virus (FMV) enhancer, carnation efflorescence ring virus (CERV) enhancer, cassava vein mosaic virus (CsVMV) enhancer, mirabilis mosaic virus (MMV) enhancer, cestrum yellow leaf curl virus (CmYLCV) enhancer, cotton leaf curl Multan virus (CLCuMV) enhancer, commelina yellow mottle virus (CoYMV) enhancer and peanut chlorotic leaf streak virus (PCLSV) enhancer.

For applications in the monocotyledon, introns include, but are not limited to, maize hsp70 intron, maize ubiquitin intron, Adh intron 1, sucrose synthase intron or rice Act1 intron. For applications in the dicotyledon, introns include, but are not limited to, CAT-1 intron, pKANNIBAL intron, PIV2 intron and “super ubiquitin” intron.

The terminators may be signal sequences suitable for polyadenylation and functioning in plants, include but are not limited to, polyadenylation signal sequences derived from nopaline synthase (NOS) gene of Agrobacterium tumefaciens, from protease inhibitor II (pin II) gene, from pea ssRUBISCO E9 gene and from α-tubulin gene.

The “effective connections” described in the present invention means the connections of nucleic acid sequences and the connections allow sequences to provide desired functions for connected sequences. The “effective connections” described in the present invention may be the connection between promoters and sequences of interest, and whereby the transcription of the sequences of interest is controlled and regulated by the promoters. When the sequences of interest encode proteins and the expression of the proteins is desired, the “effective connections” means the promoters are connected with the sequences in such a way that makes the resulting transcripts translated with a high efficiency. If the connections of the promoters and the coding sequences result in fusion transcripts and the expression of the encoded proteins is desired, such connections allow that the start codon of the resulting transcripts is the initial codon of the coding sequences. Alternatively, if the connections of promoters and coding sequences result in fusion translations and the expression of the proteins is desired, such connections allow the first start codon contained in the 5′ untranslated sequences to be connected with the promoters, and the resulted translation products to be in frame relative to the open reading frames of the desired proteins. Nucleic acid sequences for “effective connections” include, but are not limited to, sequences providing genes with expression function, i.e., gene expression elements, such as promoters, 5′ untranslated region, introns, protein-coding regions, 3′ untranslated regions, polyadenylation sites and/or transcription terminators; sequences providing DNA transfer and/or integration, i.e., T-DNA border sequences, recognition sites of site-specific recombinase, integrase recognition sites; sequences providing selection, i.e., antibiotic resistance markers, biosynthetic genes; sequences providing a scoring markers and assisting operations in vitro or in vivo, i.e., multilinker sequences, site-specific recombination sequences; and sequences providing replication, i.e., bacterial origins of replication, autonomously replicating sequences and centromere sequences.

The “pesticide” described in the present invention means that it is toxic to crop pests, including but not limited to Athetis lepigone.

In some embodiments of the present invention, Cry1F protein exhibits cytotoxicity to Athetis lepigone. For example, the transgenic plants, such as maize, in which their genomes contain exogenous DNA comprising nucleotide sequences encoding Cry1F protein, can lead to growth suppression and eventual death of Athetis lepigone by their contact with the protein after ingestion of plant tissues. Growth suppression can be lethal or sub-lethal. In some embodiments, the plants can be morphologically normal and can be cultured by conventional methods for the consumption and/or generation of products. In some instances, the transgenic plants can basically terminate the usage of chemical or biological pesticides that are Cry1F-targeted for Athetis lepigone.

The expression level of pesticidal crystal proteins (ICP) in plant tissues can be determined by any suitable methods in the art, e.g., quantification of mRNA encoding the pesticidal proteins by specific primers, or direct quantification of pesticidal proteins.

Various tests can be applied for determining the pesticidal effects of ICP in plants. One target of some embodiments of the present invention is Athetis lepigone.

In certain aspects of the present invention, the Cry1F protein may have the amino acid sequences shown as SEQ ID NO: 1 and/or SEQ ID NO: 2 in the sequence list. In addition to the Cry1F protein-coding region, other components, such as regions encoding a selection marker protein, can also be included in some aspects.

Moreover, the expression cassette comprising the nucleotide sequence encoding Cry1F protein can, in certain aspects, additionally express at least one more gene encoding herbicide resistance proteins. The herbicide resistance genes can include, but are not limited to, glufosinate resistance genes, such as bar gene and pat gene; phenmedipham resistance genes, such as pmph gene; glyphosate resistance genes, such as EPSPS gene; bromoxynil resistance genes; sulfonylurea resistance genes; herbicide dalapon resistance genes; cyanamide resistance genes; or glutamine synthetase inhibitor resistance genes such as PPT, thereby obtaining transgenic plants having both high pesticidal activity and herbicide resistance.

In some embodiments, foreign DNA is introduced into plants, for example, genes or expression cassettes or recombinant vectors encoding the Cry1F protein are introduced into plant cells. Conventional transformation methods include, but are not limited to, Agrobacterium-mediated transformation, micro-emitting bombardment, direct DNA uptake of protoplasts, electroporation, or silicon whisker mediated DNA introduction.

Some embodiments of the present invention provide a method for controlling pests, with one or more of the following advantages:

1. Internal control. Existing technologies are mainly through external actions, i.e. external factors to control the infestation of Athetis lepigone, such as the agricultural control method and the chemical control method. While some embodiments of the present invention is through Cry1F produced in plants to kill Athetis lepigone and subsequently control Athetis lepigone, i.e., through internal factors to control.

2. No pollution and no residue. The chemical control method in the art plays a certain role in the control of Athetis lepigone, but it brings pollution, destruction and residues to people, livestock and farmland ecosystem. The method for controlling Athetis lepigone of some embodiments of the present invention can eliminate the above adverse consequences.

3. Control throughout the growth period. The methods for controlling Athetis lepigone in the art are staged, while some embodiments of the present invention provides plants with the protection throughout their growth period. That is, the transgenic plants (with Cry1F) from germination, growth, until flowering, fruiting can, in some instances, avoid the damage from Athetis lepigone.

4. Control of whole individual plants. The methods for controlling Athetis lepigone in the art, for example foliar spray, are mostly localized. While some embodiments of the present invention provides a protection for the whole individual plants, for example, the roots, leaves, stems, tassels, ears, anthers, filaments, etc. of the individual transgenic plants (with Cry1F) are resistant to Athetis lepigone.

5. Stable effects. The current methods of pesticide spray require direct spraying to the surface of the crops, that is likely to cause heterogeneous spray or no spray. Some embodiments of the present invention generate plants expressing the Cry1F protein with constantly level in vivo. Also, the transgenic plants (Cry1F protein) can, in some instances, have a consistently stable effect of controlling in different locations, different time and different genetic backgrounds.

6. Simple, convenient and economical. Due to the particular stealth occurrence and damage of Athetis lepigone, its monitoring and prevention is difficult, causing a substantially increased planting cost. In contrast, some embodiments of the present invention only need transgenic plants that express Cry1F protein, thus it saves a lot of manpower, materials and financial resources.

7. Complete effect. Methods for controlling Athetis lepigone in the art are not completely efficient, and only slightly reduce the damage. In contrast, the transgenic plants (with Cry1F) in some embodiments of the present invention can lead to 100% death of the newly hatched larvae of Athetis lepigone. For example, the rare larvae can survive, but they are very small due to obvious underdevelopment or even stopping development and hardly cause any damage to the maize plants.

Some embodiments of the present invention will be described in detail through the following drawings and examples.

### EXAMPLES

The following examples illustrate some embodiments of the present invention of the methods for pest control.

### Example 1

### Acquisition and Synthesis of Cry1Fa Gene

I. Acquiring the Nucleotide Sequences of Cry1Fa

The amino acid sequence (605 amino acids) of pesticidal protein Cry1Fa-01 is shown as SEQ ID NO: 1 in the sequence list; the nucleotide sequence (1818 nucleotides) of Cry1Fa-01 encoding said amino acid sequence (605 amino acids) of pesticidal protein Cry1Fa-01 is shown as SEQ ID NO: 3 in the sequence list.

The amino acid sequence (1148 amino acids) of pesticidal protein Cry1Fa-02 is shown as SEQ ID NO: 2 in the sequence list; the nucleotide sequence (3447 nucleotides) of Cry1Fa-02 encoding the amino acid sequence (1148 amino acids) of pesticidal protein Cry1Fa-02 is shown as SEQ ID NO: 4 in the sequence list.

II. Acquiring Nucleotide Sequences of Cry1Ab and Vip3A

The nucleotide sequence (1848 nucleotides) of Cry1Ab encoding the amino acid sequence (615 amino acids) of pesticidal protein Cry1Ab is shown as SEQ ID NO: 5 in the sequence list; the nucleotide sequence (2370 nucleotides) of Vip3A encoding the amino acid sequence (789 amino acids) of pesticidal protein Vip3A is shown as SEQ ID NO: 6 in the sequence list.

III. Synthesis of the Above-Mentioned Nucleotide Sequences

The nucleotide sequences of Cry1Fa-01 (shown as SEQ ID NO: 3 in the sequence list), Cry1Fa-02 (shown as SEQ ID NO: 4 in the sequence list), Cry1Ab (shown as SEQ ID NO: 5 in the sequence list) and Vip3A (shown as SEQ ID NO: 6 in the sequence list) were synthesized by Nanjing GenScript Ltd.; the 5′ end of the synthesized nucleotide sequence of the Cry1Fa-01 (SEQ ID NO: 3) was connected to restriction site of AscI, 3′ end of the synthesized nucleotide sequence of the Cry1Fa-01 (SEQ ID NO: 3) which was connected to restriction site of BamHI; the 5′ end of the synthesized nucleotide sequence of the Cry1Fa-02 (SEQ ID NO: 4) was connected to restriction site of AscI, 3′ end of the synthesized nucleotide sequence of the Cry1Fa-02 (SEQ ID NO: 4) which was connected to restriction site of BamHI; the 5′ end of the synthesized nucleotide sequence of the Cry1Ab (SEQ ID NO: 5) was connected to restriction site of NcoI, 3′ end of the synthesized nucleotide sequence of the Cry1Ab (SEQ ID NO: 5) which was connected to restriction site of SwaI; the 5′ end of the synthesized nucleotide sequence of the Vip3A (SEQ ID NO: 6) was connected to restriction site of ScaI, 3′ end of the synthesized nucleotide sequence of the Vip3A (SEQ ID NO: 6) which was connected to restriction site of SpeI.

### Example 2

### Construction of Recombinant Expression Vectors and Transformation the Same into Agrobacterium

I. Constructing Recombinant Cloning Vectors Comprising Cry1F Gene

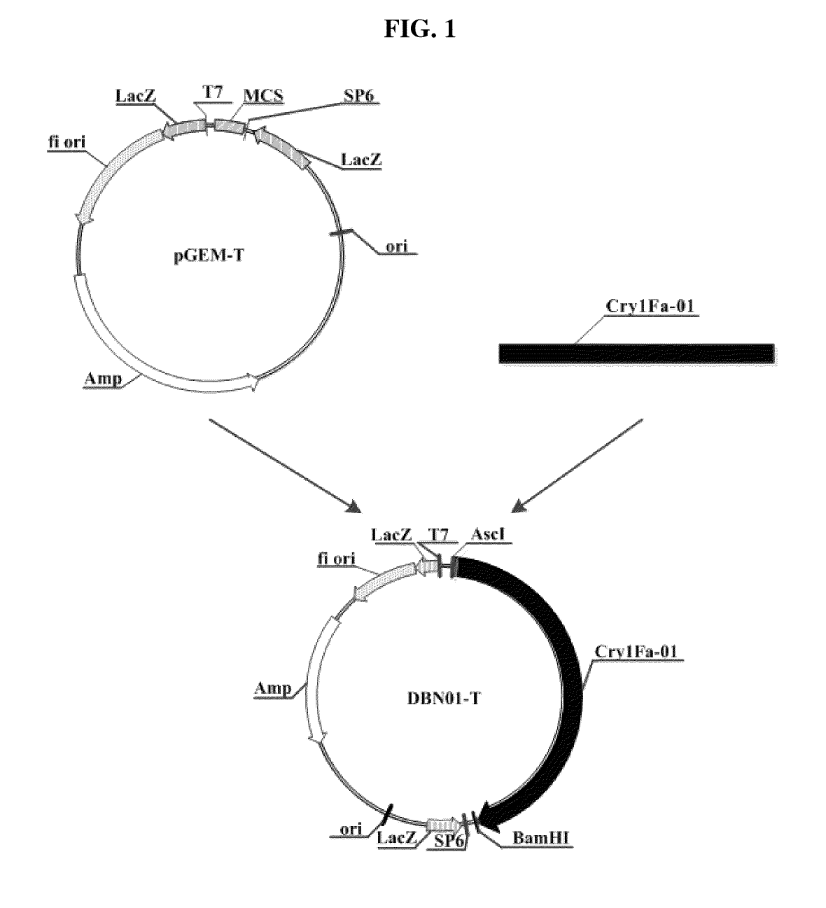

As shown in FIG. 1, the synthesized nucleotide sequence of Cry1Fa-01 was ligated with cloning vector pGEM-T (Promega, Madison, USA, CAT: A3600) according to manufacturer's protocol to generate the recombinant cloning vector DBN01-T. (Note: Amp represents Ampicillin resistance gene; f1 on represents the replication origin of phage f1; LacZ represents the start codon of LacZ; SP6 represents the promoter of SP6 RNA polymerase; T7 represents the promoter of T7 RNA polymerase; Cry1Fa-01 represents the nucleotide sequence of Cry1Fa-01 (SEQ ID NO: 3); and MCS represents a multi-cloning site).

The next step was to transform the recombinant cloning vector DBN01-T into competent cells T1 of E. coli (Transgen, Beijing, China, CAT: CD501) through a heat-shock method. Specifically, 50 μA of competent cells T1 of E. coli were mixed with 10 μl plasmid DNA (the recombinant cloning vector DBN01-T), incubated in a water bath at 42° C. for 30 seconds and then in a water bath at 37° C. for 1 hour (in a shaker at 100 rpm). The mixture was then grown overnight on a LB plate (tryptone 10 g/L, yeast extract 5 g/L, NaCl 10 g/L, agar 15 g/L, pH value was adjusted to 7.5 with NaOH) with Ampicillin (100 mg/l), of which the surface was coated with IPTG (isopropyl-thio-β-D-galactoside) and X-gal (5-bromo-4-chloro-3-indolyl-β-D-galactoside). White colonies were picked up and cultured further at 37° C. overnight in LB medium (tryptone 10 g/L, yeast extract 5 g/L, NaCl 10 g/L, Ampicillin 100 mg/L, pH value was adjusted to 7.5 with NaOH). The plasmids were extracted by an alkaline method. Specifically, the cultured bacteria in the medium were centrifuged at 12000 rpm for 1 min. The supernatant was discarded and the precipitated cells were resuspended in 100 μl ice-cold solution I (25 mM Tris-HCl, 10 mM EDTA (ethylenediamine tetraacetic acid), 50 mM glucose, pH8.0). Following the addition of 150 μl of freshly prepared solution II (0.2 M NaOH, 1% SDS (sodium dodecyl sulfate)), the tube was inverted for four times and placed on ice for 3-5 min. 150 μl ice-cold solution III (4 M potassium acetate, 2 M acetic acid) was added to the mixture, mixed immediately and thoroughly, and then placed on ice for 5-10 min, followed by centrifugation at 12000 rpm for 5 min at 4° C. The supernatant was added into 2 volumes of anhydrous ethanol, mixed thoroughly and then incubated for 5 min at room temperature. The mixture was centrifuged at 12000 rpm for 5 min at 4° C. and the supernatant was discarded. The pellet was washed with 70% (V/V) ethanol and then air dried, followed by addition of 30 μl of TE (10 mM Tris-HCl, 1 mM EDTA, PH 8.0) which contained RNase (20 μg/ml) to dissolve the pellet and digest RNA in a water bath at 37° C. for 30 minutes. The plasmids obtained were stored at −20° C. before use.

AscI and BamHI were used to identify the extracted plasmids, and positive clones were further verified by sequencing. The results showed that, the nucleotide sequence inserted into the recombinant cloning vector DBN01-T was Cry1Fa-01 shown as SEQ ID NO: 3 in the sequence list, indicating the proper insertion of the nucleotide sequence of Cry1Fa-01.

Using the above-described method for the construction of the recombinant cloning vector DBN01-T, the synthesized nucleotide sequence of Cry1Fa-02 was ligated with cloning vector pGEM-T to generate the recombinant cloning vector DBN02-T; Cry1Fa-02 is the nucleotide sequence of Cry1Fa-02 (SEQ ID NO: 4). Enzymatic digestion and sequencing were used to verify the proper insertion of the nucleotide sequence Cry1Fa-02 in the recombinant cloning vector DBN02-T.

Using the above-mentioned construction method of the recombinant cloning vector DBN01-T, the synthetic nucleotide sequence of Cry1Ab was ligated with cloning vector pGEM-T to generate the recombinant cloning vector DBN03-T; Cry1Ab is the nucleotide sequence of Cry1Ab (SEQ ID NO: 5). Enzymatic digestion and sequencing were used to verify the proper insertion of the nucleotide sequence Cry1Ab in the recombinant cloning vector DBN03-T.

Using the above-mentioned method for the construction of the recombinant cloning vector DBN01-T, the synthesized nucleotide sequence of Vip3A was ligated with cloning vector pGEM-T to generate the recombinant cloning vector DBN04-T; Vip3A is the nucleotide sequence of Vip3A (SEQ ID NO: 6). Enzymatic digestion and sequencing were used to verify the proper insertion of the nucleotide sequence of Vip3A in the recombinant cloning vector DBN04-T.

II. Constructing Recombinant Expression Vectors Comprising Cry1F Gene

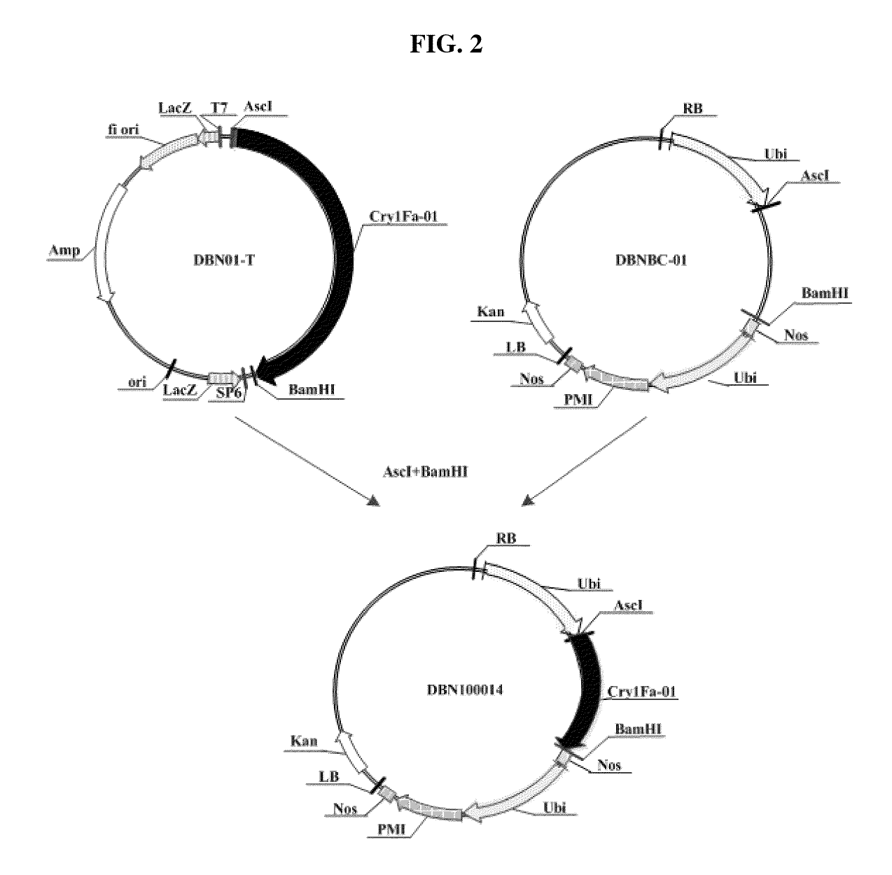

As shown in FIG. 2, the recombinant cloning vector DBN01-T and expression vector DBNBC-01 (Vector backbone: pCAMBIA2301 (available from CAMBIA institution)) were digested respectively by the restriction enzymes AscI and BamHI; the resulting fragment of the nucleotide sequence of Cry1Fa-01 was then inserted into the digested expression vector DBNBC-01 between AscI and BamHI sites to generate the recombinant expression vector DBN100014. (Note: Kan represents Kanamycin gene; RB represents right border; Ubi represents the promoter of maize ubiquitin gene (SEQ ID NO: 7); Cry1Fa-01 represents the nucleotide sequence of Cry1Fa-01 (SEQ ID NO: 3); Nos represents the terminator of nopaline synthase gene (SEQ ID NO: 8); PMI represents phosphomannose isomerase gene (SEQ ID NO: 9); and LB represents left border).

The recombinant expression vector DBN100014 was transformed into competent cells T1 of E. coli through a heat-shock method. Specifically, 50 μl competent cells T1 of E. coli were mixed with 10 μl plasmid DNA (the recombinant expression vector DBN1000124), incubated in a water bath at 42° C. for 30 seconds and then in a water bath at 37° C. for 1 hour (in a shaker at 100 rpm). The mixture was then grown at 37° C. for 12 hours on a LB plate (tryptone 10 g/L, yeast extract 5 g/L, NaCl 10 g/L, agar 15 g/L, pH value was adjusted to 7.5 with NaOH) with 50 mg/L Kanamycin. White colonies were picked up and cultured further at 37° C. overnight in LB medium (tryptone 10 g/L, yeast extract 5 g/L, NaCl 10 g/L, Kanamycin 50 mg/L, pH value was adjusted to 7.5 with NaOH). The plasmids were extracted by an alkaline method. Enzymatic digestion with AscI and BamHI was used to identify the extracted plasmids, and positive clones were further verified by sequencing. The results showed that the nucleotide sequence inserted into the recombinant cloning vector DBN0100124 between AscI and BamHI sites was Cry1Fa-01 shown as SEQ ID NO: 3 in the sequence list.

As the above method for the construction of the recombinant expression vector DBN100014, the recombinant cloning vectors DBN01-T and DBN03-T were enzymatically digested by AscI and BamHI, NcoI and SwaI respectively to generate the nucleotide sequences of Cry1Fa-01 and Cry1Ab, which were inserted into expression vector DBNBC-01 to obtain the recombinant expression vector DBN100012. As verified by enzymatic digestion and sequencing, the recombinant expression vector DBN100012 included the nucleotide sequences of Cry1Fa-01 and Cry1Ab shown as SEQ ID NO: 3 and SEQ ID NO: 5 in the sequence list.

As the above method for the construction of the recombinant expression vector DBN100014, the recombinant cloning vectors DBN02-T and DBN04-T were enzymatically digested by AscI and BamHI, ScaI and SpeI respectively to generate the nucleotide sequences of Cry1Fa-02 and Vip3A, which were further inserted into expression vector DBNBC-01 to obtain the recombinant expression vector DBN100276. As verified by enzymatic digestion and sequencing, the recombinant expression vector DBN100276 included the nucleotide sequences of Cry1Fa-02 and Vip3A shown as SEQ ID NO: 4 and SEQ ID NO: 6 in the sequence list.

III. Recombinant Expression Vectors were Transformed into Agrobacterium

The correctly constructed recombinant expression vectors, DBN100014, DBN100012 and DBN100276, were transformed into Agrobacterium LBA4404 (Invitrgen, Chicago, USA; Cat No: 18313-015) through a liquid nitrogen method. Specifically, 100 μL Agrobacterium LBA4404 and 3 μL plasmid DNA (the recombinant expression vectors) were placed in liquid nitrogen for 10 minutes, followed by incubation in a water bath at 37° C. for 10 minutes. The transformed Agrobacterium LBA4404 were inoculated in a LB tube and then cultured at 28° C., 200 rpm for 2 hours. Subsequently, the culture was applied to a LB plate containing 50 mg/L Rifampicin and 100 mg/L Kanamycin until positive individual colonies grew. The individual colonies were picked for further culture to extract plasmids. The recombinant expression vectors were identified by enzymatic digestion, that is, the recombinant expression vectors DBN100014 and DBN100012 were digested with the restriction enzymes AhdI and XbaI, while the recombinant expression vector DBN100276 was digested with restriction enzymes AhdI and AatII indicating the correct construction of the recombinant expression vectors, DBN100014, DBN100012 and DBN100276.

### Example 3

### Acquisition and Verification of Maize Plants Transformed with Cry1F Genes

I. Generation of Maize Plants Transformed with Cry1F Genes

According to the conventional method of Agrobacterium infection, the sterile cultured immature embryos of Maize Z31 were cultured with Agrobacterium strains obtained in Example 2 at section III. The T-DNAs (comprising the promoter sequence of maize Ubiquitin gene, the nucleotide sequence of Cry1Fa-01, the nucleotide sequence of Cry1Fa-02, the nucleotide sequence of Cry1Ab, the nucleotide sequence of Vip3A, PMI gene and the terminator sequence of Nos) of the recombinant expression vectors DBN100014, DBN100012 and DBN100276, which were constructed in Example 2 at section II, were transferred into the maize genome to generate the maize plants transformed with the nucleotide sequence of Cry1Fa-01, the maize plants transformed with the nucleotide sequence of Cry1Fa-01-Cry1Ab and the maize plants transformed with the nucleotide sequence of Cry1Fa-02-Vip3A. The wild-type maize plants were used as control.

The process of Agrobacterium-mediated transformation of maize was performed, as briefly described as follows. The immature embryos isolated from the maize were contacted with the Agrobacterium suspension, whereby the nucleotide sequences of Cry1Fa-01, Cry1Fa-01-Cry1Ab and/or Cry1Fa-02-Vip3A were delivered into at least one cell of either immature embryo by Agrobacterium (step 1: Infection). In this step, the immature embryos were, in some instances, immersed in Agrobacterium suspension (OD₆₆₀=0.4-0.6, infection medium (MS salt 4.3 g/L, MS vitamins, casein 300 mg/L, sucrose 68.5 g/L, glucose 36 g/L, Acetosyringone (AS) 40 mg/L, 2,4-dichlorophenoxyacetic acid (2,4-D) 1 mg/L, pH 5.3)) to initiate inoculation. The immature embryos were cultured with Agrobacterium strains for a period of time (3 days) (step 2: Co-culture). In some instances, after the step of infection, the immature embryos were cultured on a solid medium (MS salt 4.3 g/L, MS vitamins, casein 300 mg/L, sucrose 20 g/L, glucose 10 g/L, Acetosyringone (AS) 100 mg/L, 2,4-dichlorophenoxyacetic acid (2,4-D) 1 mg/L, agar 8 g/L, pH 5.8). After the co-culture step, a “recovery” step is optional, wherein there is at least an antibiotic known as inhibiting the growth of Agrobacterium (Cephalosporins) and no selection agents for plant transformants in the recovery medium (MS salt 4.3 g/L, MS vitamins, casein 300 mg/L, sucrose 30 g/L, 2,4-dichlorophenoxyacetic acid (2,4-D) 1 mg/L, agar 8 g/L, pH 5.8) (step 3: Recovery). In some instances, the immature embryos were cultured on the solid medium with an antibiotic but without selection agents to eliminate Agrobacterium and provide a recovery period for transformed cells. Next, the inoculated immature embryos were cultured on the medium with a selection agent (mannose) and the growing transformed calluses were selected (step 4: Selection). In some instances, the immature embryos were cultured on a solid selection medium with a selection agent (MS salt 4.3 g/L, MS vitamins, casein 300 mg/L, sucrose 5 g/L, mannose 12.5 g/L, 2,4-dichlorophenoxyacetic acid (2,4-D) 1 mg/L, agar 8 g/L, pH 5.8), which resulted in a selective growth of transformed cells. Further, the calluses regenerated into plants (step 5: Regeneration). In some instances, the calluses grown on the medium with the selection agent were cultured on a solid medium (MS differentiation medium and MS rooting medium) to regenerate plants.

The selected resistant calluses were transferred onto the MS differentiation medium (MS salt 4.3 g/L, MS vitamins, casein 300 mg/L, sucrose 30 g/L, 6-benzyladenine 2 mg/L, mannose 5 g/L, agar 8 g/L, pH 5.8), and cultured for differentiation at 25° C. The differentiated seedlings were transferred onto the MS rooting medium (MS salt 2.15 g/L, MS vitamins, casein 300 mg/L, sucrose 30 g/L, indole-3-acetic acid 1 mg/L, agar 8 g/L, pH 5.8), and cultured at 25° C. till the height of about 10 cm. The seedlings were then transferred into a greenhouse and grew to fructify. During the culture in the greenhouse, the seedlings were incubated at 28° C. for 16 hours and then incubated at 20° C. for 8 hours each day.

II. Verification of Maize Plants Transformed with Cry1F Genes by TaqMan Method

Using about 100 mg of leaves from each of the maize plants transformed with the nucleotide sequence of Cry1Fa-01, the maize plants transformed with the nucleotide sequence of Cry1Fa-01-Cry1Ab and the maize plants transformed with the nucleotide sequence of Cry1Fa-02-Vip3A as samples, the genomic DNA was extracted with DNeasy Plant Maxi Kit of Qiagen, and the copy numbers of Cry1F gene, Cry1Ab gene and Vip3A gene were determined by fluorescence quantitative PCR method with Taqman probe. The wild-type maize plants were analyzed as control according to the above-mentioned method. The experiments were repeated for 3 times and the results were averaged.

The detailed protocol for determining the copy numbers of Cry1F gene, Cry1Ab gene and Vip3A gene was as follows:

Step 11: 100 mg of the leaves from each of the maize plants transformed with the nucleotide sequence of Cry1Fa-01, of the maize plants transformed with the nucleotide sequence of Cry1Fa-01-Cry1Ab and of the maize plants transformed with the nucleotide sequence of Cry1Fa-02-Vip3A, and that of the wild-type maize plants were sampled and homogenized in a mortar with liquid nitrogen. Each sample was taken in triplicate.

Step 12: The genomic DNA of the above-mentioned samples was extracted with DNeasy Plant Maxi Kit of Qiagen, and the detailed method refers to the manufacturer's protocol.

Step 13: NanoDrop 2000 (Thermo Scientific) was employed to measure genomic DNA concentrations of the above-mentioned samples.

Step 14: The concentrations of genomic DNA of the above-mentioned samples were adjusted to the same concentrations in a range of 80-100 ng/μl.

Step 15: The copy numbers of the samples were determined by a fluorescence quantitative PCR method with Taqman probe. A sample that had a known copy number was used as standard, and a sample from the wild-type maize plants was used as control. Each sample was taken in triplicate and the results were averaged. The primers and probes used in the fluorescence quantitative PCR method are as follows.

The following primers and probes were used for detecting the nucleotide sequence of Cry1Fa-01:

Primer 1 (CF1): CAGTCAGGAACTACAGTTGTAAGAGGG, shown as SEQ ID NO: 10 in the sequence list;

Primer 2 (CR1): ACGCGAATGGTCCTCCACTAG, shown as SEQ ID NO: 11 in the sequence list;

Probe 1 (CP1): CGTCGAAGAATGTCTCCTCCCGTGAAC, shown as SEQ ID NO: 12 in the sequence list;

The following primers and probes were used for detecting the nucleotide sequence of Cry1Ab:

Primer 3 (CF2): TGGTGGAGAACGCATTGAAAC, shown as SEQ ID NO: 13 in the sequence list;

Primer 4 (CR2): GCTGAGCAGAAACTGTGTCAAGG, shown as SEQ ID NO: 14 in the sequence list;

Probe 2 (CP2): CGGTTACACTCCCATCGACATCTCCTTG, shown as SEQ ID NO: 15 in the sequence list;

The following primers and probes were used for detecting the nucleotide sequence of Cry1Fa-02:

Primer 5 (CF3): CAGTCAGGAACTACAGTTGTAAGAGGG, shown as SEQ ID NO: 16 in the sequence list;

Primer 6 (CR3): ACGCGAATGGTCCTCCACTAG, shown as SEQ ID NO: 17 in the sequence list;

Probe 3 (CP3): CGTCGAAGAATGTCTCCTCCCGTGAAC, shown as SEQ ID NO: 18 in the sequence list;

The following primers and probes were used for detecting the nucleotide sequence of Vip3A:

Primer 7 (CF4): ATTCTCGAAATCTCCCCTAGCG, shown as SEQ ID NO: 19 in the sequence list;

Primer 8 (CR4): GCTGCCAGTGGATGTCCAG, shown as SEQ ID NO: 20 in the sequence list;

Probe 4 (CP4): CTCCTGAGCCCCGAGCTGATTAACACC, shown as SEQ ID NO: 21 in the sequence list;

PCR reaction system:

| | |
|---|---|
| JumpStart ™ Taq ReadyMix ™ (Sigma) | 10 μl |
| 50 × mixture of primers/probes | 1 μl |
| Genomic DNA | 3 μl |
| Water (ddH₂O) | 6 μl |

The 50× mixture of primers/probes, containing 1 mM of each primer 45 μl, 100 μM of the probes 50 μl and 1×TE buffer 860 μl, was stored at 4° C. in an amber tube.

PCR conditions were as follows:

| Step | Temperature | Time |
|---|---|---|
| 21 | 95° C. | 5 min |
| 22 | 95° C. | 30 sec |
| 23 | 60° C. | 1 min |

| | |
|---|---|
| 24 | returning to step 22, repeating 40 times |

The data were analyzed by SDS2.3 software (Applied Biosystems).

As shown by the results, the nucleotide sequences of Cry1Fa-01, Cry1Fa-01-Cry1Ab and Cry1Fa-02-Vip3A were successfully integrated into the genomes of the detected maize plants respectively. The maize plants transformed with the nucleotide sequence of Cry1Fa-01, the maize plants transformed with the nucleotide sequence of Cry1Fa-01-Cry1Ab, and the maize plants transformed with the nucleotide sequence of Cry1Fa-02-Vip3A each obtained a single copy of Cry1F gene, Cry1Ab gene and/or Vip3A gene.

### Example 4

### Detection of Pesticidal Proteins in Transgenic Maize Plants

I. The Detection of the Contents of Pesticidal Proteins in Transgenic Maize Plants

The solutions involved in this experiment were as follows:

Extraction buffer: 8 g/L NaCl, 0.2 g/L KH₂PO₄, 2.9 g/L Na₂HPO₄.12H₂O, 0.2 g/L KCl, 5.5 ml/L Tween-20, pH 7.4;

Washing buffer PBST: 8 g/L NaCl, 0.2 g/L KH₂PO₄, 2.9 g/L Na₂HPO₄.12H₂O, 0.2 g/L KCl, 0.5 ml/L Tween-20, pH 7.4;

Termination solution: 1M HCl.

Three (3) mg of fresh leaves from each of the maize plants transformed with the nucleotide sequence of Cry1Fa-01, of the maize plants transformed with the nucleotide sequence of Cry1Fa-01-Cry1Ab and of the maize plants transformed with the nucleotide sequence of Cry1Fa-02-Vip3A were sampled and homogenized with liquid nitrogen, followed by the addition of 800 μl of the extraction buffer. The mixture was centrifuged at 4000 rpm for 10 min, then the supernatant was diluted 40-fold with the extraction buffer and 80 μl of diluted supernatant was used for ELISA test. ELISA (enzyme-linked immunosorbent assay) kits (ENVIRLOGIX Company, Cry1Fa, Cry1Fa/Cry1Ac and Vip3A kits) were employed to determine the ratio of the pesticidal proteins (Cry1Fa, Cry1Ab and Vip3A proteins) content divided by the weight of the fresh leaves. The detailed method refers to the manufacturer's protocol.

Meanwhile, the wild-type maize plants and the non-transgenic maize plants identified by Taqman were used as controls, and the determination followed the methods as described above. For three lines transformed with Cry1Fa-01 (S1, S2 and S3), with Cry1Fa-01-Cry1Ab (S4, S5 and S6) and with Cry1Fa-02-Vip3A (S7, S8 and S9), one line identified as non-transgenic plant (NGM) by Taqman and one line as wild type (CK), three plants for each line were used and each plant was repeated six times.

Experimental results of the pesticidal protein Cry1Fa contents in transgenic plants were shown in Table 1. Experimental results of the pesticidal protein Cry1Ab contents in transgenic plants were shown in Table 2. Experimental results of the pesticidal protein Vip3A contents in transgenic plants were shown in Table 3. The ratios of the averaged expressions of the pesticidal protein Cry1Fa divided by the weight of the fresh leaves from the maize plants transformed with the nucleotide sequences of Cry1Fa-01, Cry1Fa-01-Cry1Ab and Cry1Fa-02-Vip3A were determined as 3475.52, 3712.48 and 3888.76 respectively; the ratio of the averaged expressions of the pesticidal protein Cry1Ab divided by the weight of the fresh leaves in the maize plants transformed with the nucleotide sequence of Cry1Fa-01-Cry1Ab was 8234.7, and the ratio of the averaged expressions of the pesticidal protein Vip3A divided by the weight of the fresh leaves in the maize plants transformed with the nucleotide sequence of Cry1Fa-02-Vip3A was 3141.02. These results suggest that the transgenic maize plants have received a relatively high and stable expression of Cry1Fa protein, Cry1Ab protein and Vip3A protein.

TABLE 1
The average amount of Cry1Fa protein expressed
in the transgenic maize plants

| | Amount of Cry1Fa protein | | | Amount of Cry1Fa protein |
|---|---|---|---|---|
| | expressed in each plant (ng/g) | | | expressed in each kind of |
| | (repeated six times per plant) | | | lines (ng/g) |
| Line | 1 | 2 | 3 | Average amount (ng/g) |
| S1 | 3535.02 | 3697.34 | 2928.71 | 3475.52 |
| S2 | 3904.88 | 2808.72 | 3044.88 | |
| S3 | 3954.63 | 3572.96 | 3832.55 | |
| S4 | 3039.78 | 3600.01 | 3753.22 | 3712.48 |
| S5 | 4543.98 | 4251.25 | 3862.03 | |
| S6 | 3049.4 | 3834.01 | 3478.66 | |
| S7 | 3892.15 | 4215.07 | 3941.55 | 3888.76 |
| S8 | 3905.47 | 3816.27 | 4028.96 | |
| S9 | 3617.49 | 3795.65 | 3786.19 | |
| NGM | −0.23 | 0 | −4.21 | 0 |
| CK | −2.36 | −1.98 | 0 | 0 |

TABLE 2
The average amount of Cry1Ab protein expressed
in the transgenic maize plants

| | Amount of Cry1Ab protein | | | Amount of Cry1Ab protein |
|---|---|---|---|---|
| | expressed in each plant (ng/g) | | | expressed in each kind of |
| | (repeated six times per plant) | | | lines (ng/g) |
| Line | 1 | 2 | 3 | Average amount (ng/g) |
| S4 | 7088.4 | 9837.5 | 10626.4 | 8234.7 |
| S5 | 9866.7 | 6863.3 | 4222.4 | |
| S6 | 9912.1 | 7724.1 | 7970.9 | |
| NGM | −4.51 | −2.44 | 0 | 0 |
| CK | 0 | −6.33 | −1.97 | 0 |

TABLE 3
The average amount of Vip3A protein expressed
in the transgenic maize plants

| | Amount of Vip3A protein | | | Amount of Vip3A protein |
|---|---|---|---|---|
| | expressed in each plant (ng/g) | | | expressed in each kind of |
| | (repeated six times per plant) | | | line (ng/g) |
| Line | 1 | 2 | 3 | Average amount (ng/g) |
| S7 | 2989.67 | 3123.65 | 3176.48 | 3141.02 |
| S8 | 3205.68 | 3102.69 | 3312.03 | |
| S9 | 3059.11 | 3246.85 | 3167.95 | |
| NGM | −1.52 | 0 | −6.34 | 0 |
| CK | 0 | −0.95 | −2.31 | 0 |

II. Detection of Pest Resistance of the Transgenic Maize Plants

The maize plants transformed with the nucleotide sequence of Cry1Fa-01, the maize plants transformed with the nucleotide sequence of Cry1Fa-01-Cry1Ab and the maize plants transformed with the nucleotide sequence of Cry1Fa-02-Vip3A, the wild-type maize plants and the non-transgenic maize plants confirmed by Taqman were detected for their resistance to Athetis lepigone.

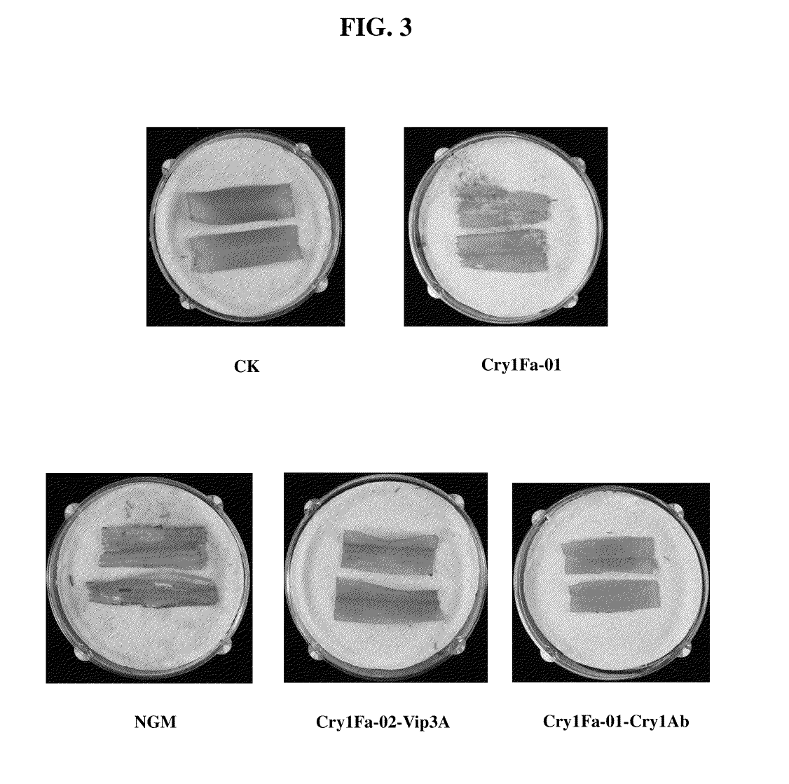

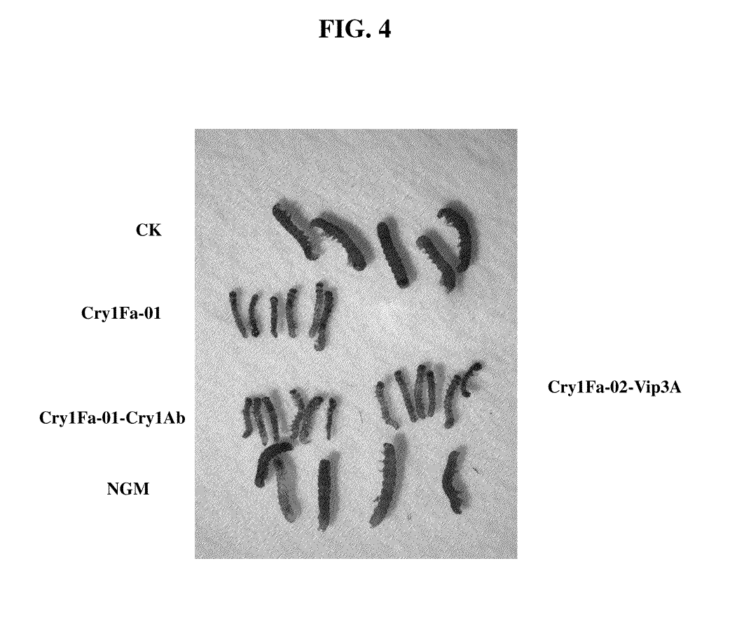

Fresh leaves of the maize plants transformed with the nucleotide sequence of Cry1Fa-01, of the maize plants transformed with the nucleotide sequence of Cry1Fa-01-Cry1Ab and of the maize plants transformed with the nucleotide sequence of Cry1Fa-02-Vip3A, and those of the wild-type maize plants and of the maize plants identified as non-transgenic plants (V3-V4 stage) by Taqman, were sampled respectively. The leaves were rinsed with sterile water and the water on the leaves was dried up by gauze. The veins of the leaves were removed, and the leaves were cut into stripes of approximately 1 cm×2 cm. Two stripes of the leaves were placed on filter paper wetted with distilled water on the bottom of round plastic Petri dishes. 10 heads of Athetis lepigone (newly hatched larvae) were putted into each dish, and the dishes with pests were covered with lids and placed at 25-28° C., relative humidity of 70%-80% and photoperiod (light/dark) 16:8 for 3 days. According to three indicators, the developmental progress, mortality and leaf damage rate of the Athetis lepigone's larvae, the resistance score was acquired: score=100×mortality+[100×mortality+90×(the number of newly hatched pests/the total number of inoculated pests)+60×(the number of newly hatched pests−the number of negative control pests/the total number of inoculated pests)+10×(the number of negative control pests/the total number of inoculated pests)]+100×(1-leaf damage rate). Three lines were transformed with Cry1Fa-01 (S1, S2 and S3), and 3 lines were transformed with Cry1Fa-01-Cry1Ab (S4, S5 and S6), and 3 lines were transformed with Cry1Fa-02-Vip3A (S7, S8 and S9), and 1 line was identified as non-transgenic plant (NGM) by Taqman, and 1 line was wild type (CK); 3 plants were chosen for test from each line, repeated six times per plants. The results were shown in Table 4, FIG. 3, and FIG. 4.

TABLE 4
The pest resistance of the transgenic maize plants inoculated with Athetis lepigone

| | | | | | Mortality of Athetis | | | |
|---|---|---|---|---|---|---|---|---|
| | | Developmental progress of | | | lepigone | | | |
| | | Athetis lepigone | | | (each line) | | | |
| | | (each line) | | | Total | | | |
| | | | Newly | | number | | | |
| | Leaf | | hatched- | | of | | Score | |
| | damage | Newly | negative | ≧negative | inoculated | Mortality | (each | |
| line | rate (%) | hatched | control | control | pests | (%) | line) | Average |
| S1 | 0 | 0 | 0 | 0 | 10 | 100 | 300 | |
| S2 | 1 | 0 | 0 | 0 | 10 | 100 | 299 | 288 |
| S3 | 1 | 3 | 0 | 0 | 10 | 70 | 266 | |
| S4 | 1 | 0.3 | 0 | 0 | 10 | 97 | 297 | |
| S5 | 0.5 | 0 | 0 | 0 | 10 | 100 | 300 | 298 |
| S6 | 1 | 0 | 0 | 0 | 10 | 100 | 299 | |
| S7 | 1 | 0.3 | 0 | 0 | 10 | 97 | 296 | |
| S8 | 1.5 | 0.6 | 0 | 0 | 10 | 94 | 292 | 288 |
| S9 | 1 | 2 | 0 | 0 | 10 | 80 | 277 | |
| NGM | 20 | 0 | 0 | 7 | 10 | 30 | 147 | 147 |
| CK | 18 | 0 | 0 | 8 | 10 | 20 | 130 | 130 |

As shown in Table 4, the scores of the maize plants transformed with the nucleotide sequence of Cry1Fa-01, of the maize plants transformed with the nucleotide sequence of Cry1Fa-01-Cry1Ab and of the maize plants transformed with the nucleotide sequence of Cry1Fa-02-Vip3A were mostly of more than 280; while the score of the wild-type maize plants was generally about 150 or less.

As shown in FIG. 3 and FIG. 4, compared with the wild-type maize plants, the maize plants transformed with the nucleotide sequence of Cry1Fa-01, the maize plants transformed with the nucleotide sequence of Cry1Fa-01-Cry1Ab and the maize plants transformed with the nucleotide sequence of Cry1Fa-02-Vip3A killed nearly 100% of the newly hatched Athetis lepigone, and inhibited the development of rarely survived larvae so that the larvae still remained in the newly hatched state after 3 days. Additionally, the maize plants transformed with the nucleotide sequence of Cry1Fa-01, the maize plants transformed with the nucleotide sequence of Cry1Fa-01-Cry1Ab and the maize plants transformed with the nucleotide sequence of Cry1Fa-02-Vip3A had little damage, shown by a small number of pinholes in a few leaves which could only be observed under a magnifier.

Thus, the maize plants transformed with the nucleotide sequence of Cry1Fa-01, the maize plants transformed with the nucleotide sequence of Cry1Fa-01-Cry1Ab, and the maize plants transformed with the nucleotide sequence of Cry1Fa-02-Vip3A all showed resistance to Athetis lepigone and was sufficient to cause adverse effects on the growth of Athetis lepigone.

The above results also appeared to show that the effective control of Athetis lepigone resulted from the Cry1F protein produced by maize plants transformed with the nucleotide sequence of Cry1Fa-01, by maize plants transformed with the nucleotide sequence of Cry1Fa-01-Cry1Ab, and by maize plants transformed with the nucleotide sequence of Cry1Fa-02-Vip3A. Similar transgenic plants that can express Cry1F can be produced to control Athetis lepigone, based on the same toxic effect of Cry1F protein to Athetis lepigone. Cry1F proteins described in the present invention include, but are not limited to, Cry1F proteins shown in the specific embodiments by the specific sequences. The transgenic plants can also generate at least one kind of additional pesticidal protein that is different from Cry1F, e.g., Cry1Ab, Cry1Ac, Cry1Ba and Vip3A.

In conclusion, some embodiments of the present invention can control Athetis lepigone by enabling the plants to produce Cry1F protein in vivo, which is toxic to Athetis lepigone. In comparison with current agricultural and chemical control methods, the method described by some embodiments of the present invention can control Athetis lepigone throughout the growth period of the plants and provide a full protection to the plants. Additionally, some aspects of the method can be one or more of stable, complete, simple, convenient, economical, pollution-free and residue-free.

Finally, it should be noted that the above embodiments merely illustrate some of the technical solutions of certain aspects of the present invention and do not limit the scope of the invention. Although some embodiments of the present invention have been described in detail, it should be appreciated that the technical solutions of the present invention can be modified or equivalently replaced without departing from the spirit and scope of the technical solutions of the invention.

What is claimed is:

1. A method for controlling Athetis lepigone, wherein the method comprises contacting Athetis lepigone with Cry1F protein.

2. The method of claim 1, wherein the Cry1F protein is Cry1Fa protein.

3. The method of claim 2, wherein the Cry1Fa protein is present in a cell that expresses the Cry1Fa protein of a plant, and Athetis lepigone contacts with the Cry1Fa protein by ingestion of the plant cell.

4. The method of claim 3, wherein the Cry1Fa protein is present in a transgenic plant that expresses the Cry1Fa protein, and Athetis lepigone contacts with the Cry1Fa protein by ingestion of a tissue of the transgenic plant; thereafter, the growth of Athetis lepigone is suppressed, and that eventually leads to Athetis lepigone's death and achieves controlling damage of Athetis lepigone to the plant.

5. The method of claim 4, wherein the transgenic plant is in any growth periods.

6. The method of claim 4, the tissue of the transgenic plant is roots, leaves, stems, tassels, ears, anthers or filaments.

7. The method of claim 4, wherein the control of the damage of Athetis lepigone to the plant does not depend on planting location.

8. The method of claim 4, wherein the control of the damage of Athetis lepigone to the plant does not depend on planting time.

9. The method of claim 4, wherein the plant is maize.

10. The method of claim 3, wherein prior to the contacting step, the method comprises a step to plant a transgenic seedling that comprises a polynucleotide encoding the Cry1Fa protein.

11. The method of claim 2, wherein the amino acid sequence of the Cry1Fa protein comprises an amino acid sequence of SEQ ID NO:1 or SEQ ID NO:2.

12. The method of claim 11, wherein the nucleotide sequence encoding the Cry1Fa protein comprises a nucleotide sequence of SEQ ID NO:3 or SEQ ID NO:4.

13. The method of claim 3, wherein the plant further expresses at least one additional nucleotide.

14. The method of claim 13, wherein the additional nucleotide encodes a Cry-like pesticidal protein, a Vip-like pesticidal protein, a protease inhibitor, lectin, α-amylase or peroxidase.

15. The method of claim 14, wherein the additional nucleotide encodes Cry1Ab protein, Cry1Ac protein, Cry1Ba protein or Vip3A protein.

16. The method of claim 15, wherein the additional nucleotide comprises a nucleotide sequence of SEQ ID NO:5 or SEQ ID NO:6.

17. The method of claim 13, wherein the additional nucleotide is dsRNA, which inhibits an important gene of a target pest.

18. A transgenic plant that expresses Cry1F protein.

19. A method of growth suppression of Athetis lepigone, wherein the method comprises contacting Athetis lepigone with the transgenic plant of claim 18 or tissues thereof.


SEQUENCE LISTING (21 sequences)

SEQ ID NO: 1 - protein, 605 aa, Artificial Sequence
  Cry1Fa-01 amino acid sequence
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLE

SEQ ID NO: 2 - protein, 1148 aa, Artificial Sequence
  Cry1Fa-02 amino acid sequence
MENNIQNQRVPYNCPNNPEVEILNEERSTGRLPLDISLSLTRFLLSEFVPGVGVAFGLFDLIWGFITPSDWSLFLLQIEQLIEQRIETLERNRAITTLRGLADSYETYIEALREREANPNNAQPREDVRIRFANTDDALITATNNFTLTSFETPLLSVYVQAANLHLSLLRDAVSFGQGWGLDIATANNHYNRLINLIHRYTKHCLDTYNQGLENLRGTNTRQWARFNQFRRDLTLTVLDTVALFPNYDVRTYPTQTSSQLTREIYTSSVIEDSPVSANIPNGFNRAEFGARPPHLTDFMNSLFVTAETVRSQTVRGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQTPLVKAHTLQSGTTVVRGPGFTGGDILRRTSGGPFAYTIVNINGQLPQRYRARIRHASTTNLRIYVTVAGERIFAGQFNKTMDTGDPLTFQSFSYATINTAFTFPMSQSSFTVGADTFSSGNEVYIDRFELIPVTATLEAESDLERAQKAVNALFTSSNQIGLKTDVTDYHIDRVSNLVECLSDEFCLDEKKELSEKVKHAKRLSDERNLLQDPNFRGINRQLDRGWRGSTDTTIQGGDDAFKENYVTLLGTSDERYPTYLYQKIDESKLKAYTRYQLRGYIEDSQDLEIYLIRYNAKHETVNVPGTGSLWPLSAPSPIGKCAHHSHHFSSDIDVGCTDLNEDLGVWAIFKIKTQDGHARLGNLEFLEEKPLVGEALARVKRAEKKWRDKREKLEWETNTVYKEAKESVDALFVNSQYDRLQADTNIAMIHAADKRVHSIREAYLPELSVIPGVNAAIFEELEGRIFTAPSLYDARNVIKNGDFNNGLSCWNVKGHVDVEEQNNHRSVPVVPEWEAEVSQEVRACPGRGYTLRVTAYKEGYGEGCVTIHEIENNTDELKFSNCVEEEVYPNNTVTCNDYTATQEEHEGTYTSRNRGYDGAYESNSSAPADYASAYEEKAYTDGRRDNPCEPNRGYGDYTPLPAGYVTKELEHLPETDKVWIEIGETEGTLIVDSVELPLMEE

SEQ ID NO: 3 - DNA, 1818 bp, Artificial Sequence
  Cry1Fa-01 nucleotide sequence
atggagaacaacatacagaatcagtgcgtcccctacaactgcctcaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagttgattgaacaaaggattgagaccttggaaaggaatcgggccatcactacccttcgtggcttagcagacagctatgagatctacattgaagcactaagagagtgggaagccaatcctaacaatgcccaactgagagaagatgtgcgtatacgctttgctaacacagatgatgctttgatcacagccatcaacaacttcacccttaccagcttcgagatccctcttctctcggtctatgttcaagctgctaacctgcacttgtcactactgcgcgacgctgtgtcgtttgggcaaggttggggactggacatagctactgtcaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacatagttgctctctttccgaactacgatgttcgtacctatccgattcaaacgtcatcccaacttacaagggagatctacaccagttcagtcattgaagactctccagtttctgcgaacatacccaatggtttcaacagggctgagtttggagtcagaccaccccatctcatggacttcatgaactctttgtttgtgactgcagagactgttagatcccaaactgtgtggggaggacacttagttagctcacgcaacacggctggcaatcgtatcaactttcctagttacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagattcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgctatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgagtaa

SEQ ID NO: 4 - DNA, 3447 bp, Artificial Sequence
  Cry1Fa-02 nucleotide sequence
atggagaacaacatacagaatcagcgcgtcccctacaactgccccaacaatcctgaagtagagattctcaacgaagagaggtcgactggcagattgccgttagacatctccctgtcccttacacgtttcctgttgtctgagtttgttccaggtgtgggagttgcgtttggcctcttcgacctcatctggggcttcatcactccatctgattggagcctctttcttctccagattgaacagctgattgaacaaaggattgagaccttggaaaggaatcgggccatcactacccttcgtggcttagcagacagctatgagacctacattgaagcactaagagagcgggaagccaatcctaacaatgcccaaccgagagaagatgtgcgtatacgctttgctaacacagatgatgctttgatcacagccaccaacaacttcacccttaccagcttcgagacccctcttctctcggtctatgttcaagctgccaacctgcacttgtcactactgcgcgacgctgtgtcgtttgggcaaggttggggactggacatagctactgccaacaatcactacaacagactcatcaatctgattcatcgatacacgaaacattgtttggatacctacaatcagggattggagaacctgagaggtactaacactcgccaatgggccaggttcaatcagttcaggagagaccttacacttactgtgttagacacagttgctctctttccgaactacgatgttcgtacctatccgactcaaacgtcatcccaacttacaagggagatctacaccagttcagtcattgaagactctccagtttctgcgaacatacccaatggtttcaacagggctgagtttggagccagaccaccccatctcacggacttcatgaactctttgtttgtgactgcagagactgttagatcccaaactgtgcggggaggacacttagttagctcacgcaacacggctggcaatcgtatcaactttcctagctacggggtcttcaatcccgggggcgccatctggattgcagatgaagatccacgtcctttctatcggaccttgtcagatcctgtcttcgtccgaggaggctttggcaatcctcactatgtactcggtcttaggggagtggcctttcaacaaactggtacgaatcacacccgcacattcaggaactccgggaccattgactctctagatgagataccacctcaagacaacagcggcgcaccttggaatgactactcccatgtgctgaatcatgttacctttgtgcgctggccaggtgagatctcaggttccgactcatggagagcaccaatgttctcttggacgcatcgtagcgctacccccacaaacaccattgatccagagagaatcactcagactcccttggtgaaggcacacacacttcagtcaggaactacagttgtaagagggccggggttcacgggaggagacattcttcgacgcactagtggaggaccattcgcgtacaccattgtcaacatcaatgggcaacttccccaaaggtatcgtgccaggatacgccatgcctctactaccaatctaagaatctacgttacggttgcaggtgaacggatctttgctggtcagttcaacaagacaatggataccggtgatccacttacattccaatctttctcctacgccactatcaacaccgcgttcacctttccaatgagccagagcagtttcacagtaggtgctgataccttcagttcaggcaacgaagtgtacattgacaggtttgagttgattccagttactgccacactcgaggcagagtctgacttggaaagagcacagaaggcggtgaatgctctgttcacttcgtccaatcagattgggctcaagacagatgtgactgactatcacatcgatcgcgtttccaaccttgttgagtgcctctctgatgagttctgtttggatgagaagaaggagttgtccgagaaggtcaaacatgctaagcgacttagtgatgagcggaacttgcttcaagatcccaactttcgcgggatcaacaggcaactagaccgtggatggaggggaagtacggacaccaccattcaaggaggtgatgatgcgttcaaggagaactatgtcacgctcttgggtacctctgacgagcgctatccaacatacctgtaccagaagatagatgaatcgaaactcaaagcctacacaagataccagttgagaggttacatcgaggacagtcaagaccttgagatctacctcatcagatacaacgccaaacatgagacagtcaatgtgcctgggacgggttcactctggccactttcagccccaagtcccatcggcaagtgcgcccaccactcacaccacttctcctcggacatagacgttggctgtaccgacctgaacgaagacctcggtgtgtgggcgatcttcaagatcaagactcaagatggccatgccaggctaggcaatctggagttcctagaagagaaaccacttgttggagaagccctcgctagagtgaagagggctgagaagaagtggagggacaagagagagaagttggaatgggaaacaaacactgtgtacaaagaagccaaagaaagcgttgacgctctgtttgtgaactcccagtatgataggctccaagctgataccaacatagctatgattcatgctgcagacaaacgcgttcatagcattcgggaagcttaccttcctgaacttagcgtgattccgggtgtcaatgctgctatctttgaagagttagaagggcgcatcttcactgcaccctccttgtatgatgcgaggaatgtcatcaagaatggtgacttcaacaatggcctatcctgctggaatgtgaaagggcacgtagatgtagaagaacagaacaatcaccgctctgtccctgttgttcctgagtgggaagcagaagtttcacaagaagttcgtgcctgtcccggccgtggctacactcttcgtgttaccgcgtacaaagaaggatacggagaaggttgcgtcaccatacacgagattgagaacaacaccgacgagctgaagttcagcaactgcgtcgaggaggaagtctacccaaacaacaccgtaacttgcaatgactacactgcgactcaagaggagcacgagggtacttacacttctcgcaatcgaggatacgatggagcctatgagagcaactcttctgcacccgctgactatgcatcagcctacgaggagaaggcttacaccgatggacgtagggacaacccttgcgaacctaacagaggctacggggactacacaccgttaccagccggctatgtcaccaaagagctagagcaccttccagaaaccgacaaggtttggattgagattggagaaacggaaggaacactcattgttgatagcgtggagttacctctgatggaggaataa

SEQ ID NO: 5 - DNA, 1848 bp, Artificial Sequence
  Cry1Ab nucleotide sequence
atggacaacaacccaaacatcaacgaatgcattccatacaactgcttgagtaacccagaagttgaagtacttggtggagaacgcattgaaaccggttacactcccatcgacatctccttgtccttgacacagtttctgctcagcgagttcgtgccaggtgctgggttcgttctcggactagttgacatcatctggggtatctttggtccatctcaatgggatgcattcctggtgcaaattgagcagttgatcaaccagaggatcgaagagttcgccaggaaccaggccatctctaggttggaaggattgagcaatctctaccaaatctatgcagagagcttcagagagtgggaagccgatcctactaacccagctctccgcgaggaaatgcgtattcaattcaacgacatgaacagcgccttgaccacagctatcccattgttcgcagtccagaactaccaagttcctctcttgtccgtgtacgttcaagcagctaatcttcacctcagcgtgcttcgagacgttagcgtgtttgggcaaaggtggggattcgatgctgcaaccatcaatagccgttacaacgaccttactaggctgattggaaactacaccgaccacgctgttcgttggtacaacactggcttggagcgtgtctggggtcctgattctagagattggattagatacaaccagttcaggagagaattgaccctcacagttttggacattgtgtctctcttcccgaactatgactccagaacctaccctatccgtacagtgtcccaacttaccagagaaatctatactaacccagttcttgagaacttcgacggtagcttccgtggttctgcccaaggtatcgaaggctccatcaggagcccacacttgatggacatcttgaacagcataactatctacaccgatgctcacagaggagagtattactggtctggacaccagatcatggcctctccagttggattcagcgggcccgagtttacctttcctctctatggaactatgggaaacgccgctccacaacaacgtatcgttgctcaactaggtcagggtgtctacagaaccttgtcttccaccttgtacagaagacccttcaatatcggtatcaacaaccagcaactttccgttcttgacggaacagagttcgcctatggaacctcttctaacttgccatccgctgtttacagaaagagcggaaccgttgattccttggacgaaatcccaccacagaacaacaatgtgccacccaggcaaggattctcccacaggttgagccacgtgtccatgttccgttccggattcagcaacagttccgtgagcatcatcagagctcctatgttctcatggattcatcgtagtgctgagttcaacaatatcattccttcctctcaaatcacccaaatcccattgaccaagtctactaaccttggatctggaacttctgtcgtgaaaggaccaggcttcacaggaggtgatattcttagaagaacttctcctggccagattagcaccctcagagttaacatcactgcaccactttctcaaagatatcgtgtcaggattcgttacgcatctaccactaacttgcaattccacacctccatcgacggaaggcctatcaatcagggtaacttctccgcaaccatgtcaagcggcagcaacttgcaatccggcagcttcagaaccgtcggtttcactactcctttcaacttctctaacggatcaagcgttttcacccttagcgctcatgtgttcaattctggcaatgaagtgtacattgaccgtattgagtttgtgcctgccgaagttaccttcgaggctgagtactga

SEQ ID NO: 6 - DNA, 2370 bp, Artificial Sequence
  Vip3A nucleotide sequence
atgaacaagaacaacaccaagctctccacacgggcacttccctcctttattgactactttaatggcatctatgggtttgctacggggatcaaggacattatgaacatgatcttcaagacagacactggcggggatcttacgctcgacgagattcttaagaatcagcaactcctgaacgatatctctggcaagctggacggcgtgaatgggtcacttaacgacctcatcgctcaggggaatctcaacacagaactgtctaaggagatcctcaagattgcaaatgagcagaaccaagttcttaatgatgtgaacaataagctcgacgccatcaacacaatgcttcgcgtgtacctcccaaagattactagcatgctctcggacgtcatgaagcagaactacgcgctgtcccttcaaattgagtatctgagcaagcagcttcaagaaatctcggacaagctggatatcattaatgtgaacgtcctcatcaacagcaccctgacggagattacaccggcgtaccagaggatcaagtatgtgaatgagaagttcgaggaactcacttttgctacagaaacttccagcaaggtcaagaaggatggctcaccagccgacatcctggatgagcttacagaactcactgagctggcgaagtccgtgaccaagaatgacgtcgatggcttcgagttttacctgaacacgttccacgacgttatggtgggcaacaatctttttgggcggagcgctctcaagactgcatcggaactgatcaccaaggagaacgttaagacgagcggctcggaggtcgggaatgtttacaacttccttatcgtcctcaccgcactccaggcccaagcgtttctcacgctgaccacctgccgcaagctcctcggcctcgcagacatcgattacacctccatcatgaacgagcacctgaacaaggagaaggaggagttccgcgtgaatatccttccgacactctcgaacactttttctaatccaaactacgctaaggtcaagggctccgacgaagatgcaaagatgatcgttgaggccaagcctggccatgcgctcatcgggttcgagatttctaacgactcaattaccgtgctgaaggtctacgaggcgaagctcaagcagaattatcaagtggacaaggattctctgtcagaggttatctacggcgacatggataagctgctttgccctgatcagtccgagcaaatctactatacgaacaatattgtcttccccaacgaatacgtgatcaccaagattgactttacgaagaagatgaagacactccggtacgaggtgacggctaacttctatgattcgtctacgggcgagatcgacctcaacaagaagaaggtcgaatcatccgaggccgaatacagaaccctgtcggcgaacgacgatggcgtgtatatgcctcttggggtcatttctgagaccttcctcacgcccatcaatggctttgggctccaggcagatgagaactcccgcctgatcacccttacgtgcaagagctacctcagggagctgctgcttgccaccgacctctctaacaaggaaacgaagctgatcgttccgccatcaggcttcatctccaatattgtggagaacgggtcaattgaggaagataatctggaaccgtggaaggctaacaataagaacgcatacgttgaccacacaggcggggtgaatggcactaaggcgctctatgtgcataaggatggtggcatctcccagttcattggcgacaagctgaagccgaagacagaatacgtgattcaatatactgtgaagggcaagccaagcatccacctcaaggatgagaacacagggtacatccattacgaagatactaacaacaacctggaggactaccagacaatcaataagaggttcacaactggcactgacctgaagggggtctatcttattctcaagtcccagaatggcgatgaggcctggggcgacaacttcatcattctcgaaatctcccctagcgagaagctcctgagccccgagctgattaacaccaataactggacatccactggcagcacgaatatctcggggaacaccctgacgctttaccagggcgggagaggcattctgaagcagaacctccaactggattcgttctctacctacagagtctatttttcagtttccggcgacgcgaatgtgcgcatcaggaactcgcgggaagtcctcttcgagaagagatacatgtctggcgctaaggatgtgtcagaaatgttcaccacgaagtttgagaaggacaacttttatatcgaactgtcccaagggaataacctctacggcggccccattgttcatttttacgacgtgagcatcaagtga

SEQ ID NO: 7 - DNA, 1992 bp, Artificial Sequence
  Promoter of Maize Ubiquitin gene
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 8 - DNA, 253 bp, Artificial Sequence
  Terminator of nopaline synthase gene
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 9 - DNA, 1176 bp, Artificial Sequence
  Phosphomannose isomerase gene
atgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 10 - DNA, 27 bp, Artificial Sequence
  Primer 1
cagtcaggaactacagttgtaagaggg

SEQ ID NO: 11 - DNA, 21 bp, Artificial Sequence
  Primer 2
acgcgaatggtcctccactag

SEQ ID NO: 12 - DNA, 27 bp, Artificial Sequence
  Probe 1
cgtcgaagaatgtctcctcccgtgaac

SEQ ID NO: 13 - DNA, 21 bp, Artificial Sequence
  Primer 3
tggtggagaacgcattgaaac

SEQ ID NO: 14 - DNA, 23 bp, Artificial Sequence
  Primer 4
gctgagcagaaactgtgtcaagg

SEQ ID NO: 15 - DNA, 28 bp, Artificial Sequence
  Probe 2
cggttacactcccatcgacatctccttg

SEQ ID NO: 16 - DNA, 27 bp, Artificial Sequence
  Primer 5
cagtcaggaactacagttgtaagaggg

SEQ ID NO: 17 - DNA, 21 bp, Artificial Sequence
  Primer 6
acgcgaatggtcctccactag

SEQ ID NO: 18 - DNA, 27 bp, Artificial Sequence
  Probe 3
cgtcgaagaatgtctcctcccgtgaac

SEQ ID NO: 19 - DNA, 22 bp, Artificial Sequence
  Primer 7
attctcgaaatctcccctagcg

SEQ ID NO: 20 - DNA, 19 bp, Artificial Sequence
  Primer 8
gctgccagtggatgtccag

SEQ ID NO: 21 - DNA, 27 bp, Artificial Sequence
  Probe 4
ctcctgagccccgagctgattaacacc